(19) United States
(12) Patent Application Publication
(10) Pub. No.: US 2011/0224576 A1
(43) Pub. Date: Sep. 15, 2011

(54) METHODS AND DEVICES FOR TISSUE COLLECTION AND ANALYSIS

(75) Inventors: George W. Jackson, Pearland, TX (US); Charles Houssiere, Houston, TX (US)

(73) Assignee: BIOTEX, INC., Houston, TX (US)

(21) Appl. No.: 13/046,738

(22) Filed: Mar. 12, 2011

Related U.S. Application Data
(60) Provisional application No. 61/313,131, filed on Mar. 12, 2010.

(51) Int. Cl. A61B 10/02

(52) U.S. Cl. 600/567

(57) ABSTRACT

The present invention is directed to methods and devices for tissue collection and analysis, and particularly to methods and devices for collecting, preserving and analyzing biopsy samples. In one aspect, a method for collecting a tissue sample includes disposing a collection device proximate and/or within a tissue, such as of a body, drawing in at least a portion of the tissue into the collection device, adhering the at least a portion of the tissue to at least a portion of the collection device and separating the sample and collection device from the remainder of the tissue and/or body. In general, the method of adhering the tissue sample to the collection device may also preserve the tissue sample, such as, for example, by altering the temperature of the tissue sample. In one embodiment, the method of adhering the tissue sample to the collection device includes lowering the temperature of the collection device and thus the tissue sample such that the tissue sample may adhere to the collection device and may also be preserved against degradation.

### CROSS-REFERENCE TO RELATED APPLICATIONS

This application claims the benefit of U.S. provisional patent application Ser. No. 61/313,131, filed Mar. 12, 2010, entitled “Methods and devices for tissue collection and analysis”, the entire contents of which are hereby incorporated by reference.

### FIELD OF THE INVENTION

This invention relates to methods and devices for tissue collection and analysis, and particularly to methods and devices for collecting, preserving and analyzing biopsy samples.

### BACKGROUND OF THE INVENTION

Biopsy is an important procedure used for the diagnosis of patients with cancerous tumors, pre-malignant conditions, and other diseases and disorders. Typically, in the case of cancer, when the physician establishes by means of procedures such as palpation, mammography or x-ray, or ultrasound imaging that suspicious circumstances exist, a biopsy is performed. The biopsy will help determine whether the cells are cancerous, the type of cancer, and what treatment should be used to treat the cancer. Biopsy may be done by an open or percutaneous technique. Open biopsy, which is an invasive surgical procedure using a scalpel and involving direct vision of the target area, removes the entire mass (excisional biopsy) or a part of the mass (incisional biopsy). Percutaneous biopsy, on the other hand, is usually done with a needle-like instrument through a relatively small incision, blindly or with the aid of an imaging device, and may be either a fine needle aspiration (FNA) or a core biopsy. In FNA biopsy, individual cells or clusters of cells are obtained for cytologic examination and may be prepared such as in a Papanicolaou smear. In core biopsy, as the term suggests, a core or fragment of tissue is obtained for histologic examination which may be done via a frozen section or paraffin section. One important area where biopsies are performed is the diagnosis of breast tumors. Definitive pathological diagnosis of tumor has traditionally been based on histological examination of invasive tissue biopsies. Therefore present instruments for biopsy have been designed to remove tissue samples while preserving morphological features useful for histopathologic classification. These traditional diagnostics have recently been augmented with immunochemical analyses for protein biomarkers which promise to further differentiate or augment disease classification and refine treatment approaches. And even more recently, an emerging milieu of tumor analysis and screening techniques based on microarray or RT-PCR analysis of RNA expression and/or other factors has been evolving.

### SUMMARY OF THE INVENTION

The present invention is directed to methods and devices for tissue collection and analysis, and particularly to methods and devices for collecting, preserving and analyzing biopsy samples. In one aspect, a method for collecting a tissue sample includes disposing a collection device proximate and/or within a tissue, such as of a body, drawing in at least a portion of the tissue into the collection device, adhering the at least a portion of the tissue to at least a portion of the collection device and separating the sample and collection device from the remainder of the tissue and/or body. In general, the method of adhering the tissue sample to the collection device may also preserve the tissue sample, such as, for example, by altering the temperature of the tissue sample. In one embodiment, the method of adhering the tissue sample to the collection device includes lowering the temperature of the collection device and thus the tissue sample such that the tissue sample may adhere to the collection device and may also be preserved against degradation.

In an exemplary embodiment, the collection device includes a coring biopsy needle and a cooling element. In general, a coring biopsy needle may be a substantially linear, hollow cylindrical tube including a lumen which may further include a sharp and/or other cutting edge at a distal end. The cutting edge may generally be utilized to aid in disrupting tissue such that the distal needle end may be positioned proximate to and/or within a target tissue. The cooling element may generally include a probe which may be disposed within the lumen of the coring biopsy needle. The cooling element may thus be positioned proximate to and/or within a target tissue within the coring biopsy needle.

In one embodiment, the cooling element includes a Joule-Thomson effect cryogenic probe. The cryogenic probe may generally include an inner structure having at least two lumens which may be connected at a distal end of the probe. In general, one lumen may be substantially smaller in cross-sectional area than the at least one other lumen. A gas and/or fluid may then be fed through the smaller of the lumens such that it may travel down the lumen to the distal end of the probe, where it may enter an enlarged space formed by the connection with the larger lumen. In general and without being bound by any particular explanation or theory, the Joule-Thomson effect may cause the gas and/or fluid to cool as it may expand into the larger lumen space and thus may cool the cryogenic probe and/or surrounding area. The cooling element may also include a penetrating tip, which may be disposed at or proximate to the distal end of the coring needle. This may be utilized to aid in penetrating through tissue to position the coring needle and cooling element within the tissue. The cooling element may further occupy a substantial portion of the cross-sectional area of the coring needle lumen.

In another aspect, a method for collecting a tissue sample includes inserting a coring biopsy needle into a tissue or body and positioning the distal end of the coring needle proximate to and/or within a target tissue. A cooling element may further be disposed within the lumen of the coring needle such that it may be positioned along with the distal end of the coring needle proximate to and/or within the target tissue. Alternatively, the cooling element may also be inserted into the lumen of the coring needle after it is positioned. In one embodiment, the cooling element may be disposed within the lumen of the coring needle when it may be positioned within a target tissue. The cooling element may then be at least partially withdrawn away from the distal end of the coring needle into the lumen of the coring needle. In general, withdrawing the cooling element into the lumen of the coring needle may produce a suction force on the tissue which may generally draw the tissue into the lumen of the coring needle. A vacuum may also be utilized to draw tissue into the lumen. The cooling element may then be utilized to cool at least the tissue drawn into the lumen, which may generally cause the tissue to adhere to the cooling element and/or the coring needle. The cooling may also generally preserve the sample of the tissue against at least some forms of degradation, such as, for example, against thermal degradation of biomolecules in the sample. The sample may then be removed from the rest of the tissue and/or the body. In one embodiment, the cooling element may be completely withdrawn from the lumen of the coring needle along with the sample which may be adhered to the cooling element. Another cooling element may also be introduced into the lumen of the coring needle such that an additional sample may be collected. This may be desirable as a new introduction of the coring needle may not be necessary. In another embodiment, the sample, cooling element and coring needle may all be withdrawn from the tissue and/or body at once. In general, the cooling element may utilize any appropriate method of cooling, such as above. Further in general, it may be desirable for the cooling to be rapid such that, for example, the time of the biopsy procedure may be reduced and/or the sample may be appropriately preserved for analysis.

In further aspect, a method for collecting a tissue sample also includes a method for preserving the sample for analysis after collection. In one embodiment, the cooled sample may be, for example, quickly transferred to a preservation device. The preservation device may, for example, maintain a low temperature and/or preserve the biomolecular and/or biochemical makeup of the sample. In one embodiment, a sample may be preserved in a chemical reagent mixture which may also be maintained at a low temperature, such as, for example, by storage in a cooling device. The sample may then be maintained for later analysis or, for example, the chemical reagent mixture and/or the preservation device may also be at least a portion of an analysis system.

The present invention together with the above and other advantages may best be understood from the following detailed description of the embodiments of the invention illustrated in the drawings.

### BRIEF DESCRIPTION OF THE FIGURES

FIG. 1 illustrates a cross-section of a biopsy device in an embodiment of the present invention;

FIG. 1a illustrates a perspective cut-away of a biopsy device in an embodiment of the present invention;

FIG. 2 illustrates a perspective see-through view of a biopsy device in an embodiment of the present invention;

FIG. 3 illustrates a vessel and a storage device in an embodiment of the present invention.

### DETAILED DESCRIPTION OF THE INVENTION

The detailed description set forth below is intended as a description of the presently exemplified device provided in accordance with aspects of the present invention and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The present invention is directed to methods and devices for tissue collection and analysis, and particularly to methods and devices for collecting, preserving and analyzing biopsy samples. In one aspect, a method for collecting a tissue sample includes disposing a collection device proximate and/or within a tissue, such as of a body, drawing in at least a portion of the tissue into the collection device, adhering the at least a portion of the tissue to at least a portion of the collection device and separating the sample and collection device from the remainder of the tissue and/or body. In general, the method of adhering the tissue sample to the collection device may also preserve the tissue sample, such as, for example, by altering the temperature of the tissue sample.

In one embodiment, the method of adhering the tissue sample to the collection device includes lowering the temperature of the collection device and thus the tissue sample such that the tissue sample may adhere to the collection device and may also be preserved against degradation.

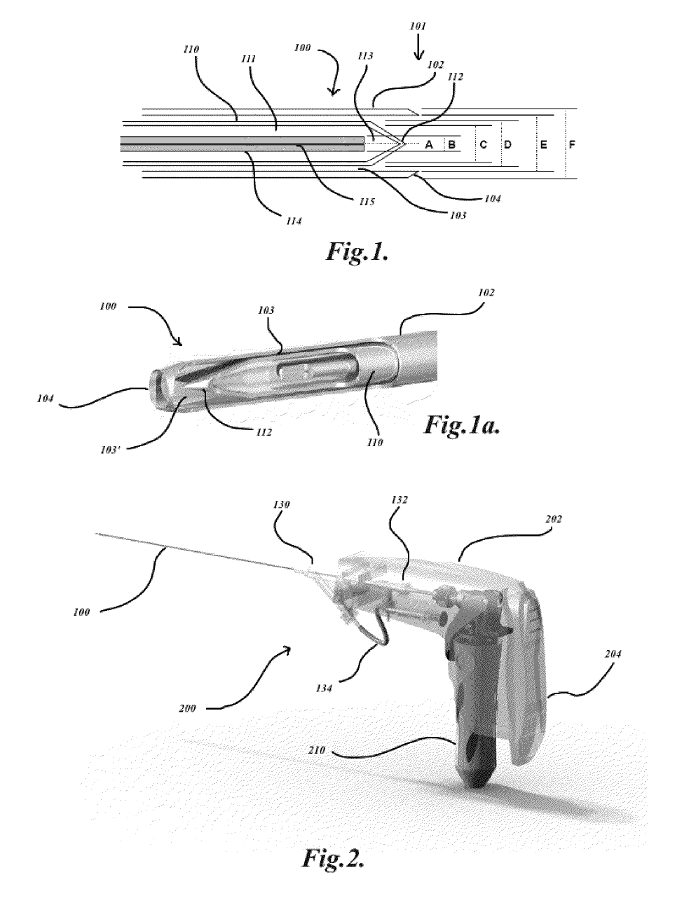

In an exemplary embodiment, as illustrated in FIGS. 1 and 1a, a collection device 100 includes a coring biopsy needle 102 and a cooling element 110. In general, a coring biopsy needle 102 may be a substantially linear, hollow cylindrical tube including a lumen 103 with an inner diameter E and an outer diameter F and which may further include a sharp and/or other cutting edge 104 at a distal end 101. The coring needle 102 may further be coated and/or treated such that it may no substantially adhere to tissue, for example, to facilitate positioning and removal of the coring needle 102 in a tissue. For example, the coring needle 102 may be coated with Teflon® or PTFE and/or any other appropriate material or combination thereof. The cutting edge 104 may generally be utilized to aid in disrupting tissue such that the distal needle end 101 may be positioned proximate to and/or within a target tissue. In one embodiment, the coring needle 102 may include, for example, a 12 gauge coring biopsy needle. The cooling element 110 may generally include a probe which may be disposed within the lumen 103 of the coring biopsy needle 102. The cooling element 110 may thus be positioned proximate to and/or within a target tissue within the coring biopsy needle 102.

In one embodiment, the cooling element 110 includes a Joule-Thomson effect cryogenic probe, as illustrated in FIGS. 1 and 1a. The cryogenic probe may generally include an inner structure having at least two lumens 111, 115 which may be connected at a distal end chamber 113 of the cooling element 110. In general, one lumen, such as lumen 115, may be substantially smaller in cross-sectional area than the at least one other lumen, such as lumen 111, as illustrated. A gas and/or fluid may then be fed through the smaller of the lumens, such as 115, such that it may travel down the lumen to the distal end chamber 113 of the cooling element 110, where it may enter an enlarged space formed by the connection with the larger lumen 111.

In general and without being bound to any particular explanation or theory, the Joule-Thomson effect may cause the gas and/or fluid to cool as it may expand into the larger lumen space, which may thus cool the cryogenic probe and/or surrounding area. Expansion of an ideal gas under constant enthalpy conditions (i.e. the gas does no work) is adiabatic, meaning that the temperature remains constant. In a non-ideal gas, however, Van der Waals forces between gas molecules play two roles in the energy of the gas. As a gas expands, the average distance between molecules increases leading to an increase in potential energy with respect to attractive forces. At the same time, increased intermolecular distance leads to a decrease in molecular collisions which leads to a decrease in average potential energy for the gas since molecular collisions lead to a temporary increase in potential energy. Because the total energy must be conserved, a change in potential energy leads to a change in kinetic energy and hence the temperature of the gas. The direction of temperature change depends on which of the two processes dominates and is characterized by the Joule-Thomson coefficient μ_{J-T} defined in the equation:

$${μ}_{J - T} = {\frac{\partial P}{\partial T} }_{Δ H = 0} \vert$$

In one embodiment, the cooling element 110 may utilize the Joule-Thomson expansion of CO₂, such as supplied from small disposable “pellet gun” cylinders. Carbon dioxide may be desirable as it may be stored under pressure as a liquid at room temperature. Any other appropriate gas and/or fluid may also be utilized, such as, for example, nitrogen. The cooling capacity for the system may be calculated from the Joule-Thomson coefficient and the expected pressure drop. For example, assuming a 100× cross-sectional expansion between the smaller lumen 115 and the chamber 113, the chamber 113 may result in an exit pressure of approximately one atmosphere, the pressure change from the vapor pressure of liquid CO₂ at 25° C. to atmospheric may result in a temperature change of ΔT=μ_{J-T}ΔP=(1.11 K/atm)(1 atm−63 atm)=−70K. For further example, to achieve an end temperature of −20° C. in a sample of approximately 0.1 g of tissue, the enthalpy change required may be calculated from ΔH=mCₚΔT and is given by (−20-37)° C. (3.5 J/g K)(0.1 g)=20 J. Assuming an exit gas temperature of 0° C., the required mass of CO₂ is given by 20 J/[(45 K) (0.86 J/g K)]=0.5 g. A typical small cylinder may contain 4 g of gas.

The cooling element 110 may also include a penetrating tip 112, which may be disposed at or proximate to the distal end 101 of the coring needle 102. This may be utilized to aid in penetrating through tissue to position the coring needle 102 and cooling element 110 within the tissue. The cooling element 110 may further occupy a substantial portion of the cross-sectional area of the coring needle lumen 103 and may generally be translatable longitudinally in the lumen 103. The cooling element 110 may then be withdrawn into the lumen 103 to vary the size of the distal lumen portion 103′, as illustrated in FIG. 1a.

An apparatus for collecting a tissue sample may also be included, such as the apparatus 200 in FIG. 2. The apparatus 200 may house a device 100, such as shown in FIGS. 1 and 1a. The device 100 may include a coupling 130 which may connect the cooling element 110 to a gas and/or fluid supply valve 132 and an exhaust valve 134. The supply valve 132 may be utilized to provide gas and/or fluid to the cooling element 110, as above, from a gas and/or fluid supply 210 within the housing 202 of the apparatus 200. The supply 210 may be actuated utilizing a trigger 204. Exhaust gas and/or fluid may then be vented to the atmosphere via exhaust valve 134.

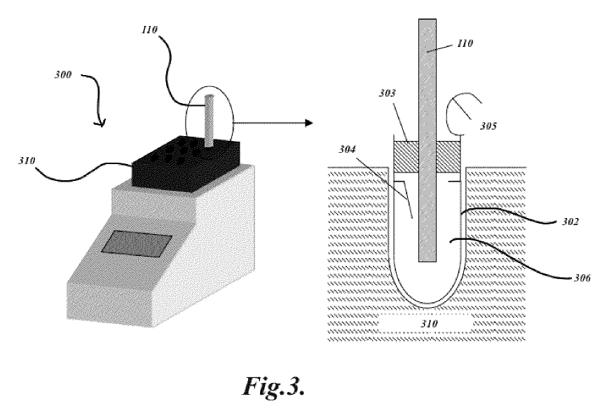

In another aspect, a method for collecting a tissue sample includes inserting a coring biopsy needle 102 into a tissue or body and positioning the distal end 101 of the coring needle 102 proximate to and/or within a target tissue. A cooling element 110 may further be disposed within the lumen 103 of the coring needle 102 such that it may be positioned along with the distal end 101 of the coring needle 102 proximate to and/or within the target tissue. Alternatively, the cooling element 110 may also be inserted into the lumen 103 of the coring needle 102 after it is positioned. In one embodiment, the cooling element 110 may be disposed within the lumen 103 of the coring needle 102 when it may be positioned within a target tissue. The cooling element 110 may then be at least partially withdrawn away from the distal end 101 of the coring needle 102 into the lumen 103 of the coring needle 102. In general, withdrawing the cooling element 110 into the lumen 103 of the coring needle 102 may produce a suction force on the tissue which may generally draw the tissue into the distal portion 103′ of the lumen 103 of the coring needle 102. A vacuum and/or other form of negative pressure may also be applied to aid, supplement or replace the suction force of drawing the cooling element 110 into the lumen 103. A vacuum and/or other form of negative pressure may, for example, be applied at a valve and/or other interface that may be in fluid contact with lumen 103. For example, exhaust valve 134 may be in fluid contact with lumen 103 and a vacuum and/or negative pressure source may be connected to the exhaust valve 134. The cooling element 110 may then be utilized to cool at least the tissue drawn into the distal lumen portion 103′, which may generally cause the tissue to adhere to the cooling element 110 and/or the lumen 103 of the coring needle 102. The cooling may also generally preserve the sample of the tissue against at least some forms of degradation, such as, for example, against thermal degradation of biomolecules in the sample. The sample may then be removed from the rest of the tissue and/or the body. In one embodiment, the cooling element 110 may be completely withdrawn from the lumen 103 of the coring needle 102 along with the sample which may be adhered to the cooling element 110. Another cooling element 110 may also be introduced into the lumen 103 of the coring needle 102 such that an additional sample may be collected. This may be desirable as a new introduction of the coring needle may not be necessary. In another embodiment, the sample, cooling element 110 and coring needle 102 may all be withdrawn from the tissue and/or body at once. In general, the cooling element 110 may utilize any appropriate method of cooling, such as above. Further in general, it may be desirable for the cooling to be rapid such that, for example, the time of the biopsy procedure may be reduced and/or the sample may be appropriately preserved for analysis. The vessel 302 may, for example, include a prepared reagent mixture 306, which may be in a sealed manner, such as with seals 304, 305 and/or with a septum 303. The seals 304, 305 may include, for example, foil seals which may generally maintain a seal and integrity of the reagent mixture 306 until punctured by, for example, introduction of the sample, such as with the cooling element 110 as illustrated in FIG. 3. The septum 303 may be utilized, for example, to position and hold the cooling element 110 in the vessel 302.

In further aspect, a method for collecting a tissue sample also includes a method for preserving the sample for analysis after collection. In one embodiment, the cooled sample may be, for example, quickly transferred to a preservation device, such as the device 300 in FIG. 3. The preservation device may, for example, maintain a low temperature and/or preserve the biomolecular and/or biochemical makeup of the sample. The device 300 may, for example, include a cooling block 310 for holding samples from a cooling element 110 or other sample device. In one embodiment, a sample may be preserved in a chemical reagent mixture 306, such as in a reagent vessel 302, which may also be maintained at a low temperature, such as, for example, by storage in a cooling block 310. The sample may then be maintained for later analysis or, for example, the chemical reagent mixture 306 and/or the preservation device 300 may also be at least a portion of an analysis system.

In some embodiments, a method of preserving a tissue sample may generally include inhibiting degradation of tissue components, such as by utilizing inhibitors of degrading biomolecules, which may include nucleases, proteases, and/or cellular structures for degradation of biomolecules. In one embodiment, aptamers may be utilized to inhibit degrading biomolecules.

Aptamers are generally short, functional nucleic acid molecules isolated from large random libraries through a process termed systematic evolution of ligands by exponential enrichment, or SELEX. Contrary to the actual genetic material, their specificity and characteristics are not generally directly determined by their primary sequence, but instead by their tertiary structure. While aptamers are analogous to antibodies in their adaptability and range of application, they display several potential advantages over their protein counterparts. They are smaller, faster and more economical to produce, may show improved affinity and specificity, are highly biocompatible and nonimmunogenic and can easily be modified chemically to yield improved properties. For clinical applications, the fact that aptamers can be prepared fully in vitro may allow for a faster regulatory approval process. Aptamers are selected in an iterative process termed SELEX (systematic evolution of ligands by exponential enrichment). Multiple rounds of binding and PCR amplification of the binding population are used to narrow the library down to only the highest affinity nucleic acids. In contrast to many other biomedical methods, absolutely no information about the target molecules is necessary.

In general, aptamers and/or any other appropriate inhibitor may be utilized at any appropriate step of a tissue sample collection and may also be utilized in, for example, the reagent mix 306, and/or as a component of the device 100. Inhibitors may also, for example, be introduced into the tissue and/or cells of the sample. For further example, it may be desirable for inhibitors such as aptamers to be introduced into the cytosol of the sample cells such that degradation of biomolecules may be inhibited prior to the collection of the sample.

### Example of a Cryobiopsy Probe

The ‘Mark I’ version of the biopsy sampling probe includes a Joule-Thompson effect cryogenic refrigeration tip located inside of a 12 Ga (0.109″ OD) en face coring biopsy needle. The cryotip consists of a low-pressure expansion chamber formed from thin-wall 14 Ga (0.083″ OD; 0.067″ ID) Type 304 stainless steel hypodermic tubing surrounding a length of 1/32″ Type 316 stainless steel high pressure liquid chromatography (HPLC) tubing (0.03125 OD; 0.007″ ID) which serves as the throttling nozzle for the cryogenic gas. The cryotip assembly slides within the lumen of the biopsy needle body and can serve as a trocar during negotiation of the probe to the sampling site. Once in place, the cryotip is retracted into the lumen of the outer needle a few mm and negative pressure applied through the proximal communication with the needle lumen draws a sample inside at which point the cyrogenic gas is released to snap-freeze the sample within the tube. The biopsy sampling probe was fabricated using commercially available materials including 12 Ga bone biopsy needles from Remington, thin wall hypodermic tubing from Small Parts, and HPLC tubing from ThermoFischer. In the Mark I probe, thin Teflon heat-shrink tubing will be used to effect the coating on the surface of the outer needle.

### Example of Preservation of a Tissue Sample

Immediately after sample freezing, which may take about 10 seconds, the cryoprobe and needle assembly are withdrawn from the tissue. A PTFE (Teflon) coating on the outside of the biopsy probe needle prevents sticking to adjacent tissues. Once outside the body, the probe with sample still inside is placed directly into a benchtop cryostat where chemical storage will occur in the liquid phase at sub-zero temperatures. The cryostat will be based on a small footprint Peltier cooler like the EchoTHERM (or similar) which maintains temperatures down to −10° C. in 1° increments. A custom cooling tray similar to a multi-vial plate design house individual reagent mixtures pre-cooled to the prescribed temperature. Support for contaminant-free insertion of the probe device into the reagent solution is integrated into the housing removable from the chiller base plate.

It will be appreciated by those of ordinary skill in the art that the present invention can be embodied in other specific forms without departing from the spirit or essential character hereof. The present description is therefore considered in all respects to be illustrative and not restrictive. The scope of the present invention is indicated by the appended claims, and all changes that come within the meaning and range of equivalents thereof are intended to be embraced therein.

1. A biopsy device for collecting a tissue sample comprising:
a coring needle having an internal lumen; and
a cryogenic probe removable and translatably disposed within said internal lumen;
wherein said cryogenic probe is translated into said internal lumen to draw a tissue into said internal lumen and said cryogenic probe preserves and adheres to said tissue.

2. The biopsy device of claim 1, wherein said cryogenic probe comprises a Joule-Thomson effect probe.

3. The biopsy device of claim 2, wherein said Joule-Thomson effect probe comprises a pair of connected lumens, one of said lumens being significantly smaller than the other of said lumens.

4. The biopsy device of claim 3, further comprising a gas source.

5. The biopsy device of claim 1, wherein said cryogenic probe comprises a sharp point.

6. A biopsy system for collecting and preserving a sample from a tissue comprising:
a coring needle having an internal lumen;
a cryogenic probe removable and translatably disposed within said internal lumen, wherein said cryogenic probe is translated into said internal lumen to draw a tissue into said internal lumen and said cryogenic probe preserves and adheres to said tissue; and
a cryogenic preservation device for storing and preserving tissue adhered to said cryogenic probe.

7. The biopsy system of claim 6, wherein said cryogenic probe comprises a Joule-Thomson effect probe.

8. The biopsy system of claim 7, wherein said Joule-Thomson effect probe comprises a pair of connected lumens, one of said lumens being significantly smaller than the other of said lumens.

9. The biopsy system of claim 8, further comprising a gas source.

10. The biopsy system of claim 6, wherein said cryogenic probe comprises a sharp point.

11. The biopsy system of claim 6, wherein said cryogenic preservation device comprises a cooling system.

12. The biopsy system of claim 11, wherein said cooling system comprises a Peltier effect cooler.

13. The biopsy system of claim 6, further comprising a vessel for storing said tissue.

14. The biopsy system of claim 13, further comprising a reagent mixture in said vessel.

15. The biopsy system of claim 6, further comprising a negative pressure source in fluid communication to said internal lumen.

16. A method for collecting and preserving a sample from a tissue comprising:
positing a coring needle having an internal lumen proximate to a tissue;
inserting a cryogenic probe within said internal lumen proximate to said tissue;
drawing at least a portion of a tissue into said internal lumen;
cooling said at least a portion of a tissue with said cryogenic probe; and
storing and preserving said at least a portion of a tissue with a cryogenic preservation device.

17. The method of claim 16, wherein said cooling comprises flowing a gas from a first lumen to a second lumen, said first lumen being much smaller than said second lumen.

18. The method of claim 16, wherein said drawing comprises applying a negative pressure to said internal lumen.

19. The method of claim 16, wherein said storing and preserving comprises placing said at least a portion of a tissue into a reagent mixture.

20. The method of claim 16, wherein said at least a portion of a tissue adheres to said cryogenic probe.
